(19) United States
(12) Patent Application Publication
(10) Pub. No.: US 2025/0277755 A1
(43) Pub. Date: Sep. 4, 2025

(54) ROI FILTER MODULE WITH A REDUCED INSTALLATION SPACE

(71) Applicant: Siemens Healthineers AG, Forchheim (DE)

(72) Inventor: Michael PLOESSNER, Neustadt (DE)

(73) Assignee: Siemens Healthineers AG, Forchheim (DE)

(21) Appl. No.: 19/063,922

(22) Filed: Feb. 26, 2025

(30) Foreign Application Priority Data
Feb. 29, 2024 (DE) 10 2024 201 873.2

(51) Int. Cl. G01N 23/04

(52) U.S. Cl. CPC G01N 23/04; G01N 2223/1016; G01N 2223/313; G01N 2223/316; G01N 2223/321

(57) ABSTRACT

One or more example embodiments relates to an ROI filter module, a collimator, an X-ray tube assembly and an imaging modality. The inventive ROI filter module includes a filter unit including a holding apparatus and a filter disk, the filter disk connected to the holding apparatus, the filter disk including at least one cylindrical filter cutout; and a kinematic guiding apparatus configured to move the filter unit within a movement plane, the kinematic guiding apparatus including, a first swivel joint, and a second swivel joint, a rotational axis of the first swivel joint and a rotational axis of the second swivel joint are spaced apart, are aligned parallel, and are perpendicular to the movement plane, wherein the second swivel joint is rotatable about the rotational axis of the first swivel joint, and the filter unit is rotatable about the rotational axis of the second swivel joint.

### CROSS-REFERENCE TO RELATED APPLICATION(S)

The present application claims priority under 35 U.S.C. § 119 to German Patent Application No. 10 2024 201 873.2, filed Feb. 29, 2024, the entire contents of which are incorporated herein by reference.

### FIELD

One or more example embodiments relates to an ROI filter module, a collimator, an X-ray tube assembly and an imaging modality.

### RELATED ART

Conventionally, ROI filter modules are used to at most partially reduce X-rays of an X-ray source in an examination region. For this a filter disk frequently has a filter cutout by which a particular Region of Interest (ROI) can be examined without reducing the X-rays.

In the portion of the filter disk adjoining the filter cutout, by contrast, the X-rays are at least partially attenuated, so the region outside of the ROI area can typically still be seen, but frequently only in reduced image quality. As a result, a total radiation dose of a patient situated in the examination region can advantageously be reduced for a user of the X-ray source while retaining the same image quality, in particular in the ROI area.

U.S. Pat. No. 5,278,887 A involves an apparatus and a method for reducing the X-ray dose during a fluoroscopic procedure, wherein a filter element is used to selectively attenuate the X-ray radiation striking the body of a patient. The filter element enables unattenuated X-rays the mapping of a region of interest selected by a doctor and thus generates an image with higher intensity and low noise. However, the regions around the region of interest are mapped with attenuated radiation, whereby a less intensive, more noisy image results. A real-time image processing system is used to compensate the image brightness and possibly filter the compensated region in order to reduce the noise. As a result, the image quality is restored, so a doctor can orient the site of an intervention in the body of a patient and can observe the position of the region of interest relative to an environment from the perspective.

US 2012 187 312 A1 provides a radiation monitoring system and a method with which the radiation supplied to a patient and/or the operator of the device is minimized. The radiation monitoring system can be used in a large number of applications, including applications in which a radiation source is used for inspecting an object, such as in medical imaging, diagnosis and therapy, in manufacturing procedures in which radiation is used, in scanning systems in airports, in different security facilities and in the automation and process control of nuclear reactors. The radiation monitoring system and method can also be used with 3D imaging.

US 2016 192 892 A1 discloses a multi-image imaging system with the capacity for different X-ray exposure of various input regions of an image intensifier or another X-ray detector. Collimators are provided for controlling the amount of radiation in different regions of the image, and image processing arranges for displaying images of different quality. Movement methods are available to move the collimators in order to generate optimum image frames.

The X-ray image device from US 2016 211 045 A1 comprises an X-ray source which is configured such that it irradiates X-rays onto an object; an X-ray detector which is configured such that it detects X-rays irradiated by the X-ray source; and a disk, which is rotatably provided between the X-ray source and the X-ray detector, wherein Region of Interest filters (ROI filters) are configured such that they filter X-rays irradiated by the X-ray source. Openings of difference sizes are provided on the disk of the ROI filter.

The invention from US 2016 317 104 A1 relates to a multi-image imaging system which a radiation source, a detector with an input region, a monitor which is configured for displaying captured images, means for determining at least one Region of Interest (ROI) of an object on the displayed image and a collimator with means for projecting the at least one region of interest (ROI) on at least one selected part of the input surface exposed by the X-ray source. The collimator comprises at least three substantially non-overlapping plates which are mounted in a plane generally parallel to the plane of the detector input surface. Each plate comprises a first edge which is in contact with an edge of a first adjacent plate, and a second edge, which adjoins the first edge in contact with an edge of a second adjacent plate; and means for moving each individual plate.

WO 2013 132 387 A2 describes an X-ray system composed of an X-ray source, an individual substantially round collimator, a camera, a detector and a monitor, means for moving the collimator in a plane which generally runs parallel to the plane of the collimator; and the collimator comprises a central opening which allows all of the radiation through, an outer ring, which reduces the penetrating radiation by an amount which depends on the material and the thickness of the material, and an inner ring between the central opening and the outer ring, wherein the thickness changes as a function of the spacing from the center, beginning with the thickness zero on the side of the central opening and ending with the thickness of the outer annular space on the side of the outer annular space.

### SUMMARY

One challenge when using such a conventional ROI filter module is that the filter disk with the filter cutout can dwell not just stationarily in the beam path with the X-rays, but frequently has to be dynamically movable. An ROI filter module can preferably be moved in two spatial directions within a movement plane.

The inventor knows to use a kinematic guiding apparatus for this purpose, which moves the filter disk within the movement plane. The kinematic guiding apparatus typically has for this two linear rails arranged at right angles to one another. In particular, the linear rail of the second direction of movement is coupled to the carriages of the linear rail of the first direction of movement in this case. The filter disk is preferably mounted on the carriages of the linear rail of the second direction of movement. The kinematic guiding apparatus has, in particular, drive means for moving the filter disk. In particular for reasons of available installation space and/or in order to reduce the moved masses, the drive means are stationarily arranged on a base plate of the ROI filter module, with the filter disk being moved by the drive means through an expensive belt guide.

One or more example embodiments provides an ROI filter module, a collimator, an X-ray tube assembly and an imaging modality with a reduced installation space requirement.

This is achieved by the features of the independent claims. Advantageous embodiments are described in the subclaims.

### BRIEF DESCRIPTION OF THE DRAWINGS

The invention will be described and explained in more detail below with reference to the exemplary embodiments represented in the Figures. Basically, substantially unchanging structures and units will be labeled with the same reference numerals in the following description of the Figures as when the respective structure or unit appeared for the first time.

In the drawings:

FIG. 1 shows an inventive ROI filter module according to one or more example embodiments,

FIG. 2 shows a first exemplary embodiment of a filter unit,

FIG. 3 shows a second exemplary embodiment of a filter unit,

FIG. 4 shows a first exemplary embodiment of the ROI filter module,

FIG. 5 shows a second exemplary embodiment of the ROI filter module and

FIG. 6 shows an inventive imaging modality.

### DETAILED DESCRIPTION

Independent of the grammatical term usage, individuals with male, female or other gender identities are included within the term.

The inventive ROI filter module has
a filter unit and
a kinematic guiding apparatus for moving the filter unit within a movement plane,
wherein the filter unit has a holding apparatus and a filter disk, connected to the holding apparatus, with at least one cylindrical filter cutout,
   characterized in that
the kinematic guiding apparatus has a first swivel joint and a second swivel joint,
wherein a rotational axis of the first swivel joint and a rotational axis of the second swivel joint are spaced apart from one another and aligned parallel and are perpendicular to the movement plane,
wherein the second swivel joint is mounted so it can rotate about the rotational axis of the first swivel joint and
wherein the filter unit is mounted so it can rotate about the rotational axis of the second swivel joint.

The inventive ROI filter module is advantageous, in particular, because compared to the conventional embodiment with linear rails, a much lower installation space requirement emanates from the swivel joints and typically the range of functions of the ROI filter module simultaneously remains the same. Advantageously, the ROI filter module can thus also be used when there is less installation space available. Alternatively or in addition, the saved installation space can be used for further functions and/or modules. Overall a base plate, which is essential for the conventional embodiment with linear rails, can primarily preferably be omitted or have smaller dimensions.

A further advantage of the ROI filter module relates to its decreased complexity owing to the reduced number of components. Furthermore, in particular the total mass moved via the swivel joints is preferably decreased, so the drive means, in particular, can be dimensioned to be smaller and/or to be less powerful. In a counter action, in particular the movement speed within the movement plane can be increased.

It is also advantageous that a distinct cost advantage can be achieved, typically due the omission of the linear rails. In particular, the swivel joints and/or the ball bearings required for them are frequently significantly less expensive than the linear rails.

The ROI filter module is configured, in particular, to at most partially reduce X-rays of an X-ray source in an examination region. In other words, in particular at least the cylindrical filter cutout of the filter disk for the X-rays remains free. The ROI filter module is suitable, in particular, for a reduction to not equal to zero of X-rays of an X-ray source in an examination region. The ROI filter module can advantageously allow the X-rays to pass unhindered in a region of interest (ROI), while in an adjacent region the X-rays are partially or completely reduced. The ROI filter module is configured, in particular, to reduce X-rays with a photon energy up to 200 keV.

The ROI filter module corresponds, in particular, to an ROI assembly. The ROI filter module or the ROI assembly is typically suitable for assembly with at least one further module or a further assembly. The ROI filter module is, in particular, an apparatus.

The ROI filter module has the filter unit and the kinematic guiding apparatus. The filter unit can be moved, in particular, via the kinematic guiding apparatus. The kinematic guiding apparatus has, in particular, a fixpoint relative to which the filter unit can be moved. The kinematic guiding apparatus is configured, in particular, to move the filter unit within or parallel to the movement plane. The filter unit can be moved via the kinematic guiding apparatus in two spatial directions in particular. The movement plane is defined, in particular, by the two spatial directions.

The kinematic guiding apparatus is configured, in particular, for a dynamic movement of the filter unit. In particular, the filter unit can be moved several times via the kinematic guiding apparatus. The filter unit is stationary for a limited time, in particular, between the movement procedures via the kinematic guiding apparatus.

The movement of the filter unit comprises, in particular, a shift and/or deflection of the filter unit. The movement of the filter unit can comprise a change in the current relative position and/or the setting of a new relative position of the filter unit relative to the fixpoint of the kinematic guiding apparatus.

During movement of the filter unit, the holding apparatus and the filter disk, in particular, are moved jointly owing to their connection. The holding apparatus and the filter disk are, in particular, permanently and/or detachably connected together. The filter disk is, in particular, fixed to the holding apparatus. The holding apparatus is embodied, in particular, for holding the filter disk. The holding apparatus is typically fixed to the kinematic guiding apparatus. The filter disk is, in particular, arranged parallel to or in the movement plane.

The filter disk is preferably composed of a material opaque to X-rays. The material opaque to X-rays can be, in particular, tungsten and/or lead.

The filter disk can be a filter plate. One surface of the filter disk is typically larger by a multiple than a thickness of the filter disk. The thickness of the filter disk can be less than 50 mm, in particular less than 10 mm. The thickness can be, for example, 0.1 mm.

The filter cutout is preferably a through-hole. In this case the through-hole typically forms a hollow cylinder. Alternatively it is conceivable to embody the filter cutout as a recess. Advantageously, a cross-section of the filter cutout is round.

The filter cutout is typically centrally arranged on the filter disk. In each case the filter cutout is preferably not arranged on the edge of the filter disk. The filter cutout is advantageously arranged spaced apart from an edge of the filter disk in such a way that the X-rays cannot simultaneously propagate through the filter cutout and past the edge of the filter disk.

The filter disk can have a plurality of filter cutouts. The plurality of filter cutouts are typically spaced apart from one another in such a way that the X-rays can only propagate through one of the plurality of filter cutouts. At least two of the plurality of filter cutouts typically have a different cross-sectional area.

The first swivel joint and the second swivel joint have, in particular, independent rotational axes. In other words, the rotational axis of the first swivel joint and the rotational axis of the second swivel joint are located side by side and not one on top of the other. If the rotational axes are located side by side, they are, in particular, spaced apart from one another and advantageously aligned parallel in such a way that they are perpendicular to the movement plane. The rotational axis of the first swivel joint and the rotational axis of the second swivel joint define, in particular, the movement plane in each case. Since the rotational axis of the first swivel joint and the rotational axis of the second swivel joint are aligned parallel to one another, the defined movement planes are in each case identical or at least parallel to one another. In the latter case the two parallel movement planes define that movement plane in or parallel to which the kinematic guiding apparatus can move the filter unit.

Each swivel joint can comprise two rotary parts which can be rotated relative to one another about the rotational axis. One rotary part can be embodied as a lever arm, for example as a rotor, or alternatively as a fixpoint, for example as a stator. A fixpoint can be, for example, a base plate to which the kinematic guiding apparatus is immovably fixed. In this case the filter unit can be moved around the fixpoint of the base plate via the kinematic guiding apparatus. The base plate can be configured, in particular, for fixing a drive means of the ROI filter module and/or for fixing the ROI filter module to a further module, in particular a collimator module.

It is conceivable that, depending on the framework, a rotary part forms a rotor for one swivel joint and simultaneously a stator for a different swivel joint. Depending on the framework, in particular one rotary part is stationary and the other rotary part rotating relative to the stationary rotary part.

For example, the second swivel joint can be arranged on a rotating rotary part of the first swivel joint. In this case the second swivel joint is mounted to rotate about the rotational axis of the first swivel joint. The rotating rotary part of the first swivel joint can form the stationary rotary part of the second swivel joint, and vice versa. The first swivel joint and the second swivel joint are connected to one another in this case via a lever arm, in particular.

In particular, the holding apparatus of the filter unit can from the rotating rotary part of the second swivel joint. Alternatively, the second swivel joint can have a lever arm as the rotating rotary part, to which arm the holding apparatus is fixed.

One embodiment provides that the holding apparatus has a first supporting plate and that the filter disk is immovably arranged relative to the first supporting plate. This embodiment is advantageous, in particular, because the first supporting plate can protect the filter disk from mechanical damage and/or contamination. Alternatively or in addition, the filter unit is advantageously more rigid due to two-dimensional contact of the first supporting plate with the filter disk. The construction with the first supporting plate is frequently less complex and/or less expensive compared to the conventional embodiment with a steel frame. The first supporting plate and the filter disk preferably overlap maximally. The first supporting plate and the filter disk are preferably arranged one on top of the other.

One embodiment provides that the first supporting plate is formed from an X-ray-transparent material. This embodiment is advantageous, in particular, because, compared to the filter disk, a contribution of the first supporting plate to the reduction of the X-ray radiation is comparatively low or non-existent. This embodiment is advantageous, in particular, if the first supporting plate has a greater area than the filter disk since a section of the first supporting plate protruding beyond the filter disk can be positioned in the X-rays without attenuating the X-rays at all or without attenuating them significantly. This embodiment also advantageously makes it possible, compared with the conventional embodiment with the steel frame, for an edge region of the filter unit, in particular the holding apparatus, to be arranged in the beam path of the X-rays whereas, conventionally, the steel frame reduces the X-rays.

One embodiment provides that the X-ray-transparent material is a plastics material. This embodiment is advantageous, in particular, because plastics material frequently has a comparatively low X-ray absorption and/or sufficient strength for holding the filter disk.

One embodiment provides that the plastics material is fiber-reinforced. The fibers of the fiber-reinforced plastics material can have, in particular, carbon. One advantage of this embodiment relates to a greater strength of the first supporting plate.

One embodiment provides that the holding apparatus also has a second supporting plate, wherein the filter disk is arranged in a sandwich construction between the first supporting plate and the second supporting plate. The first supporting plate and the second supporting plate can basically be substantially identical and/or identical in construction. Typically, the first supporting plate and the second supporting plate have a symmetrical and/or congruent construction. The sandwich construction means that the filter unit has a layered construction composed of the first supporting plate, the filter disk and the second supporting plate.

One embodiment provides that the filter disk is fixed to the first supporting plate and/or to the second supporting plate by a fixing means. Alternatively or in addition the first supporting plate and the second supporting plate can be fixed to one another by a fixing means. In the latter case the filter disk, for example, can be wedged, in particular without fixing means, between the first supporting plate and the second supporting plate. The fixing means can be, in particular, at least one screw joint, rivet joint, glued joint and/or a weld joint. The fixing means can comprise a plurality of connections of this kind.

One embodiment provides that at least one section of the first supporting plate and/or the second supporting plate protrudes beyond the filter disk. The first supporting plate and the second supporting plate can, in particular, be dimensioned in such a way that they form a frame around the filter disk. This embodiment is advantageous, in particular, to make it possible for the protruding section to fix the first supporting plate and/or the second supporting plate to the kinematic guiding apparatus. For example, the fixing means can be arranged in the protruding section.

One embodiment provides that the kinematic guiding apparatus has a first drive means (first driver) for rotating the second swivel joint about the first swivel joint and a second drive means (second driver) for rotating the filter unit about the second swivel joint. The first drive means has, for example, a motor and a force transmitter. The second drive means has, for example, a motor and a force transmitter. The force transmitter is suitable, in particular, for transmitting the force provided by the motor. The force transmitter can be, in particular, a chain transmitter or a belt transmitter. In particular, the motor can provide a force which is transmitted to a swivel joint via the force transmitter in order to rotate the swivel joint about its rotational axis. This embodiment is advantageous, in particular, because the kinematic guiding apparatus can advantageously move the filter unit with motor force.

One embodiment provides that the first swivel joint has a deflection pulley and that the second drive means can be deflected via the deflection pulley of the first swivel joint. The deflection pulley can have a transmission ratio equal to 1 or not equal to 1. In particular, the force transmitter of the second drive means can be deflected by the deflection pulley. This embodiment advantageously makes it possible for the motor of the second drive means to be immovably arranged in relation to the first swivel joint and the second swivel joint. That the first swivel joint has a deflection pulley means, in particular, that a rotational axis of the deflection pulley is located on the rotational axis of the first swivel joint. The rotational axis of the deflection pulley and the rotational axis of the first swivel joint are decoupled from one another, in particular, so the deflection pulley is rotated independently of the first swivel joint, and vice versa.

An alternative embodiment to the previous embodiment provides that the second drive means has a rotary motor and that the first swivel joint has the rotary motor of the second drive means. This embodiment advantageously makes it possible for the rotary motor to be moved together with the first swivel joint. The rotary motor is embodied, in particular, to be movable. That the first swivel joint has the rotary motor means, in particular, that a rotational axis of the rotary motors is located on the rotational axis of the first swivel joint. The rotational axis of the rotary motor and the rotational axis of the first swivel joint are decoupled from one another, in particular, so the rotary motor is rotated independently of the first swivel joint, and vice versa.

An inventive depth diaphragm has
an ROI filter module
a collimator module for limiting an effective radiation cutout,
   wherein via the kinematic guiding apparatus the at least one cylindrical filter cutout of the filter disk can be swiveled-in in such a way that the cylindrical filter cutout and the effective radiation cutout overlap along a direction perpendicular to the movement plane.

The depth diaphragm is suitable, in particular, for consecutively reducing and/or limiting X-rays via a plurality of modules, in particular via the ROI filter module and of the collimator module. In other words, the depth diaphragm enables shaping of the X-rays which propagate through the depth diaphragm, in particular through the ROI filter module and the collimator module. The X-rays typically propagate perpendicular to the movement plane, in particular parallel to the direction perpendicular to the movement plane or along the direction perpendicular to the movement plane.

The collimator module is typically configured to change a size of the effective radiation cutout. The effective radiation cutout typically has a rectangular shape. The collimator module can have a plurality of lead plates for limiting the effective radiation cutout. Alternatively or in addition, the effective radiation cutout can be limited via arc covers which can be rotated relative to one another. Limiting the effective radiation cutout means, in particular, that the X-rays can propagate through the effective radiation cutout unhindered and/or unfiltered, while the X-rays outside of the effective radiation cutout are preferably completely reduced or absorbed. An area of the effective radiation cutout is typically larger by at least a factor of 1.5, preferably 2 or 4, than the area of the filter cutout of the filter disk.

The ROI filter module can be arranged above the collimator module. Alternatively, the ROI filter module can be arranged below the collimator module. In other words, the ROI filter module can be arranged between an X-ray source and the collimator module. Alternatively, the collimator module can be arranged between the X-ray source and the ROI filter module.

That the at least one cylindrical filter cutout can be swiveled-in, means, in particular, that the at least one cylindrical filter cutout can also be swiveled-out. The swiveling-in and/or out takes place, in particular, via the kinematic guiding apparatus.

Swiveling-in comprises, in particular, sliding the filter unit into the beam path of the X-rays. Swiveling-out comprises, in particular, removing the filter unit from the beam path.

Swiveling-in of the filter cutout means, in particular, activating the filter unit. Swiveling-out of the filter cutout means, in particular, deactivating the filter unit.

In particular after swiveling-in, the swiveled-in filter cutout and the effective radiation cutout typically overlap along the direction perpendicular to the movement plane. In particular after swiveling-out, the swiveled-out filter cutout and the effective radiation cutout frequently do not overlap along the direction perpendicular to the movement plane. It is conceivable that, in particular after swiveling-out, a region of the swiveled-out filter disk outside of the filter cutout and the effective radiation cutout overlap along the direction perpendicular to the movement plane. Alternatively, the filter disk can be pivoted out in such a way that the filter disk and the effective radiation cutout do not overlap along the direction perpendicular to the movement plane.

If the filter cutout is swiveled-in in such a way that the cylindrical filter cutout and the effective radiation cutout overlap along the direction perpendicular to the movement plane, X-rays can typically propagate through the filter cutout and the effective radiation cutout along this direction. Overall the free area for the unhindered passage of the X-rays is typically limited to the area of the filter cutout. Typically, X-rays, which are not absorbed by a region of the filter disk outside of the filter cutout, can propagate through the effective radiation cutout if this region overlaps the effective radiation cutout.

If the filter cutout of the filter disk, in particular the filter disk as such, is positioned so as not to overlap in relation to the effective radiation cutout, i.e. in particular is not swiveled-in, but is preferably swiveled-out, X-rays can typically propagate through the entire effective radiation cutout. The free area for the unhindered passage of the X-rays typically corresponds to the area of the effective radiation cutout, if no region of the filter disk outside of the filter cutout overlaps the effective radiation cutout.

The kinematic guiding apparatus moves the filter unit, in particular, for the swiveling-in and/or the swiveling-out of the filter cutout. That via the kinematic guiding apparatus the at least one cylindrical filter cutout of the filter disk can be swiveled-in and/or swiveled-out means, in particular, that the at least one cylindrical filter cutout of the filter disk can be moved from a start position through to a target position within the movement plane via the kinematic guiding apparatus in such a way that after movement the filter cutout and the effective radiation cutout overlap or can no longer overlap.

An inventive X-ray tube assembly has
a depth diaphragm and
an X-ray source which is aligned with the effective radiation cutout of the collimator module.

The X-ray source is typically an X-ray tube with an evacuated housing in which electrons are accelerated from a cathode toward an anode. The X-rays are typically produced when the accelerated electrons interact on the anode. For the acceleration of the electrons the X-ray source typically has an acceleration unit which can accelerate the electrons via a high voltage with a value up to 200 kV.

The X-ray source is aligned, in particular, with the effective radiation cutout in such a way that the X-rays can basically propagate through the effective radiation cutout. In particular, the X-ray source is aligned in such a way that the X-rays are perpendicular to the movement plane of the ROI filter module. The X-rays run, in particular, parallel to the rotational axis of the first swivel joint and/or to the rotational axis of the second swivel joint.

The evacuated housing can have, for example, an X-ray exit window around which a flange is arranged. For example, the depth diaphragm can be fixed to the flange. It is conceivable that the flange also comprises a lead window. Alternatively or in addition, a pre-filtering module with a copper filter can be arranged between the evacuated housing and the depth diaphragm. Typically, the depth diaphragm is aligned relative to the X-ray source in such a way that the ROI filter module is arranged between the evacuated housing and the collimator module.

An inventive imaging modality has
an X-ray tube assembly,
an X-ray detector and
an examination region arranged between the X-ray tube assembly and the X-ray detector,
   wherein X-rays of the X-ray source in the examination region can be at most partially reduced via the ROI filter module.

The imaging modality is suitable, in particular, for diagnostic imaging. Diagnostic imaging is, in particular, an angiography, a radiography, a mammography and/or a computed tomography. Alternatively or in addition, the imaging modality can be suitable for materials testing and/or a safety check.

The X-rays issuing from the X-ray tube assembly illuminate, in particular, the examination region before they strike the X-ray detector. The X-ray detector detects, in particular, attenuation profiles which characterize an examination object situated in the examination region. An image of the examination object can advantageously be reconstructed via the attenuation profiles.

The examination region is typically situated between the X-ray tube assembly and the X-ray detector. The examination object is positioned, for example, on a patient couch in the examination region. The examination object or the examination region is radiographed, in particular, by the X-rays.

The ROI filter module is suitable for reducing those X-rays of the X-ray source, which radiograph the examination region, via the filter disk. Owing to the at least one filter cutout the ROI filter module can frequently not completely, but at most only partially, reduce the X-rays in the examination region. At most partially means, in particular, to not equal to zero or by less than 100%. The ROI filter module is, in particular, not suitable for completely reducing the X-rays in the examination region. In contrast, the collimator module can typically completely reduce the X-rays in the examination region.

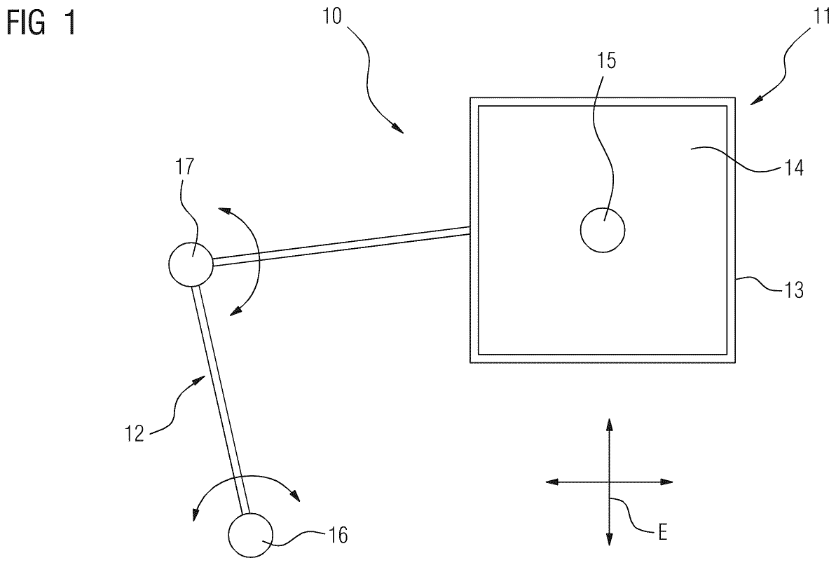

FIG. 1 shows a schematic view of the inventive ROI filter module 10 from a bird's eye view with viewing direction perpendicular to the movement plane E.

The ROI filter module 10 has a filter unit 11 and a kinematic guiding apparatus 12 for moving the filter unit 11 within a movement plane E. The filter unit 11 has a holding apparatus 13 and a filter disk 14 connected to the holding apparatus 13, with at least one cylindrical filter cutout 15.

The kinematic guiding apparatus 12 has a first swivel joint 16 and a second swivel joint 17. A rotational axis of the first swivel joint 16 and a rotational axis of the second swivel joint 17 are spaced apart from one another and aligned parallel and are perpendicular to the movement plane E. The second swivel joint 17 is mounted so it can be rotated about the rotational axis of the first swivel joint 16. The filter unit 11 is mounted so it can be rotated about the rotational axis of the second swivel joint 17.

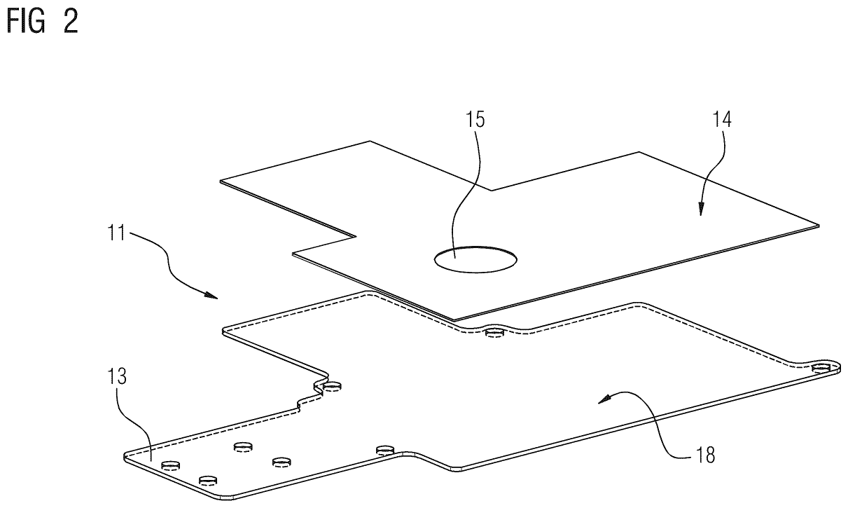

FIG. 2 shows a first exemplary embodiment of the filter unit 11 in a perspective exploded view.

The holding apparatus 13 has a first supporting plate 18. The filter disk 14 is immovably arranged relative to the first supporting plate 18. The first supporting plate 18 is made from an X-ray-transparent material, with the X-ray-transparent material being a plastics material. The plastics material can optionally be fiber-reinforced. At least one section of the first supporting plate 18 protrudes beyond the filter disk 14.

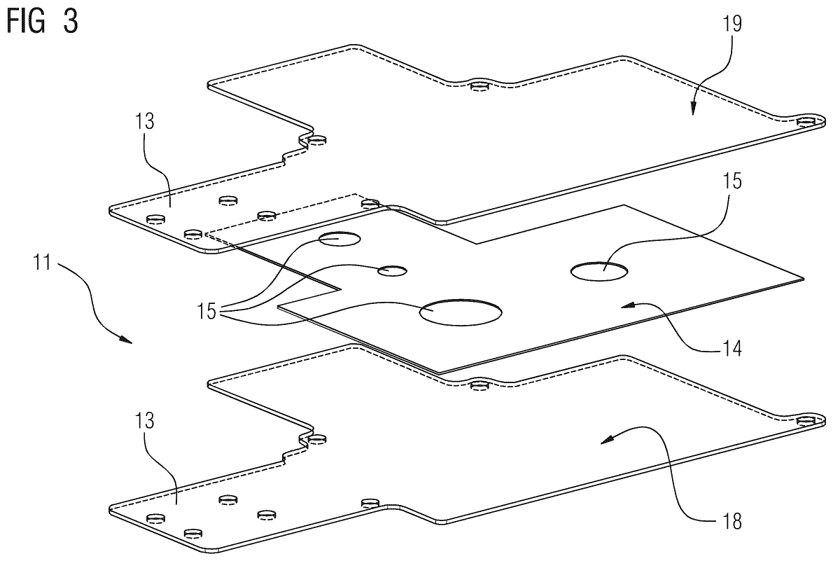

FIG. 3 shows a second exemplary embodiment of the filter unit 11 in a perspective exploded view.

The holding apparatus 13 also has a second supporting plate 19. The filter disk 14 is arranged in a sandwich construction between the first supporting plate 18 and the second supporting plate 19. The filter disk 14 in FIG. 3 has a plurality of filter cutouts 15. At least one section of the first supporting plate 18 and the second supporting plate 19 protrudes beyond the filter disk 14.

The first supporting plate 18 and the second supporting plate 19 are fixed to one another by a fixing means. Alternatively or in addition, but not shown in FIG. 3, the filter disk can be fixed to the first supporting plate and/or to the second supporting plate by a fixing means.

The fixing means of the exemplary embodiment in FIG. 3 comprises, for example, a rivet joint. Alternative embodiments are a screw joint, glued joint and/or a weld joint.

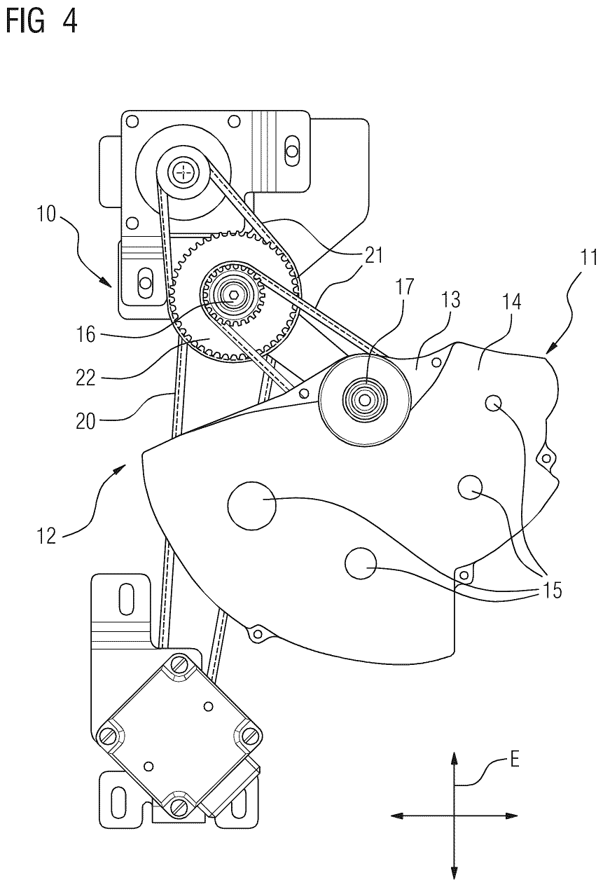

FIG. 4 shows a first exemplary embodiment of the ROI filter module 10 from a bird's eye view.

The kinematic guiding apparatus 12 has a first drive means 20 for rotating the second swivel joint 17 about the first swivel joint 16 and a second drive means 21 for rotating the filter unit 11 about the second swivel joint 16. The first swivel joint 16 has a deflection pulley 22. The second drive means 21 can be deflected by the deflection pulley 22 of the first swivel joint 16. The transmission ratio of the deflection pulley 22 is not equal to 1. The first drive means 20 and the second drive means 21 can each have a rotary motor.

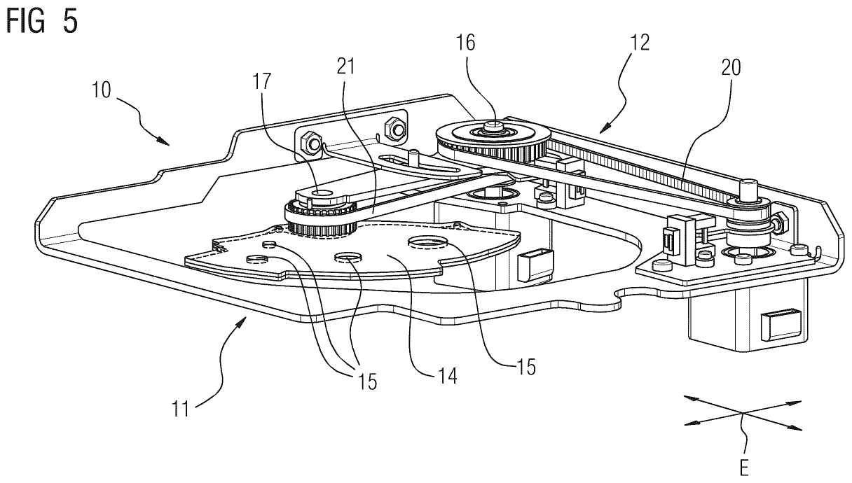

FIG. 5 shows a second exemplary embodiment of the ROI filter module 10 in a perspective view. Compared to the embodiment in FIG. 4, the first swivel joint 16 has the rotary motor of the second drive means 21 instead of the deflection pulley 22.

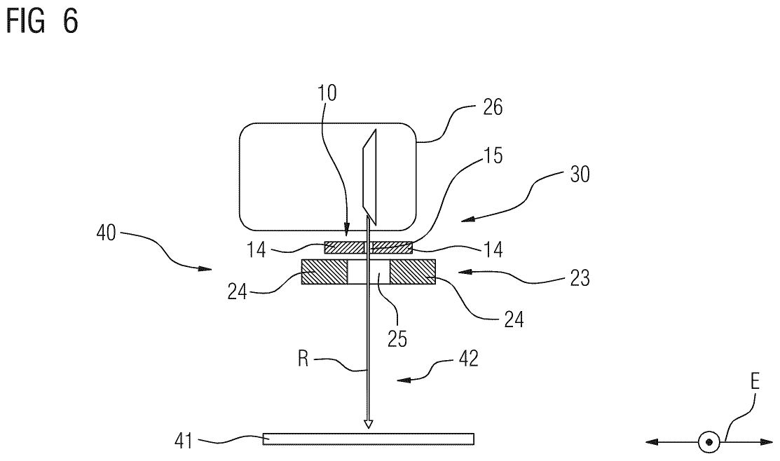

FIG. 6 shows an inventive imaging modality 40 in a section along the X-ray beam path R. The image plane of FIG. 5 is perpendicular to the movement plane E.

An inventive depth diaphragm 23 has an ROI filter module 10 and a collimator module 24 for limiting an effective radiation cutout 25. The collimator module 24 shapes the effective radiation cutout 25, in particular. The at least one cylindrical filter cutout 15 of the filter disk 14 can be swiveled-in via the kinematic guiding apparatus 12 (not shown in FIG. 6) in such a way that the cylindrical filter cutout 15 and the effective radiation cutout 25 overlap along a direction perpendicular to the movement plane E. In FIG. 5 the filter cutout 15 is swiveled-in and thus positioned to overlap the effective radiation cutout 25.

An inventive X-ray tube assembly 30 has a depth diaphragm 23 and an X-ray source 26. The X-ray source 26 is aligned with the effective radiation cutout 25 of the collimator module. The X-rays generated at the anode of the X-ray source 26 propagate along the X-ray beam path R. The X-ray beam path R is perpendicular to the movement plane E.

The imaging modality 40 has the X-ray tube assembly 30, an X-ray detector 41 and an examination region 42 arranged between the X-ray tube assembly 31 and the X-ray detector 41. X-rays of the X-ray source 26 in the examination region 42 can be at most partially reduced via the ROI filter module 10. An examination object can be arranged in the examination region 42. The examination object can be, in particular, a patient or a material or another object. The X-rays of the X-ray source 26 typically have at most a photon energy of 200 keV, typically more than 20 keV and/or less than 150 keV.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative used descriptors herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, terms all (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiments, it is nevertheless not limited by the disclosed examples and a person skilled in the art can derive other variations herefrom without departing from the scope of protection of the invention.

1. An Region of Interest (ROI) filter module, comprising:
a filter unit including a holding apparatus and a filter disk, the filter disk connected to the holding apparatus, the filter disk including at least one cylindrical filter cutout; and
a kinematic guiding apparatus configured to move the filter unit within a movement plane, the kinematic guiding apparatus including,
a first swivel joint, and
a second swivel joint, a rotational axis of the first swivel joint and a rotational axis of the second swivel joint are spaced apart, are aligned parallel, and are perpendicular to the movement plane,
wherein the second swivel joint is rotatable about the rotational axis of the first swivel joint, and
the filter unit is rotatable about the rotational axis of the second swivel joint.

2. The ROI filter module of claim 1, wherein
the holding apparatus includes a first supporting plate, and
the filter disk is immovably arranged relative to the first supporting plate.

3. The ROI filter module of claim 2, wherein the first supporting plate is formed from an X-ray-transparent material.

4. The ROI filter module of claim 3, wherein the X-ray-transparent material is a plastics material.

5. The ROI filter module of claim 4, wherein the plastics material is fiber-reinforced.

6. The ROI filter module of claim 2, wherein the holding apparatus includes a second supporting plate, wherein the filter disk is between the first supporting plate and the second supporting plate.

7. The ROI filter module of claim 6, wherein the filter disk is fixed to at least one of the first supporting plate or the second supporting plate by a fixing means.

8. The ROI filter module of claim 6, wherein the first supporting plate and the second supporting plate are fixed to one another by a fixing means.

9. The ROI filter module of claim 7, wherein the fixing means comprises at least one of a screw joint, a rivet joint, a glued joint or a weld joint.

10. The ROI filter module of claim 6, wherein at least one section of at least one of the first supporting plate or the second supporting plate protrudes beyond the filter disk.

11. The ROI filter module of claim 1, wherein the kinematic guiding apparatus includes a first driver configured to rotate the second swivel joint about the first swivel joint and a second driver configured to rotate the filter unit about the second swivel joint.

12. The ROI filter module of claim 11, wherein the first swivel joint includes a deflection pulley and the second driver is deflectable via the deflection pulley of the first swivel joint.

13. The ROI filter module of claim 11, wherein the second driver includes a rotary motor and the first swivel joint includes the rotary motor of the second driver.

14. A depth diaphragm, comprising:
the ROI filter module of claim 1; and
a collimator module configured to limit an effective radiation cutout,
wherein via the kinematic guiding apparatus the at least one cylindrical filter cutout of the filter disk is swivelable-in such that the cylindrical filter cutout and the effective radiation cutout overlap along a direction perpendicular to the movement plane.

15. An X-ray tube assembly, comprising:
the depth diaphragm of claim 14; and
an X-ray source aligned with the effective radiation cutout of the collimator module.

16. An imaging modality, comprising:
the X-ray tube assembly of claim 15;
an X-ray detector; and
an examination region between the X-ray tube assembly and the X-ray detector,
wherein X-rays of the X-ray source in the examination region are at most partially reduced via the ROI filter module.
